(19) United States
(12) Patent Application Publication
(10) Pub. No.: US 2026/0293827 A1
(43) Pub. Date: Oct. 1, 2026

(54) TOMATO PLANT THAT IS RESISTANT TO TOBAMOVIRUS AT ELEVATED TEMPERATURE

(71) Applicant: Enza Zaden Beheer B.V., Enkhuizen (NL)

(72) Inventors: Sergio De La Fuente Van Bentem, Amsterdam (NL); Albert Grit, Enkhuizen (NL); Cornelis Walter Verweij, Enkhuizen (NL)

(21) Appl. No.: 19/478,623

(22) PCT Filed: Apr. 5, 2024
(86) PCT No.: PCT/EP2024/059346

§ 371 Date: Oct. 24, 2025

(30) Foreign Application Priority Data
Apr. 26, 2023 (NL) 2034701

(51) Int. Cl. A01H 1/04; A01H 1/00; C07K 14/415; C12Q 1/6895

(52) U.S. Cl. CPC A01H 1/045; A01H 1/1225; A01H 1/126; C07K 14/415; C12Q 1/6895; C12Q 2600/13

(57) ABSTRACT

The present invention relates to a plant of the S. lycopersicum species that is resistant to Tobamovirus at temperatures of at least 28° C., wherein the plant comprises a TBRFV resistance gene and further comprises at least one temperature sensitivity reducing (TSR) gene. The present invention further relates to a combination of the TBRFV resistance gene in combination with at least one temperature sensitivity reducing (TSR) gene for providing resistance to a Tobamovirus in a S. lycopersicum plant at a temperature of at least 28° C. and methods for providing a tomato plant that is TBRFV resistant at temperatures of at least 28° C.

The present invention relates to a plant of the S. lycopersicum species that is resistant to Tobamovirus at elevated temperatures, wherein the plant comprises a TBRFV resistance gene and further comprises at least one temperature sensitivity reducing (TSR) gene. The present invention further relates to a combination of the TBRFV resistance gene in combination with at least one temperature sensitivity reducing (TSR) gene for providing resistance to a Tobamovirus in a S. lycopersicum plant at a temperature of at least 28° C. and methods for providing a tomato plant that is TBRFV resistant at temperatures of at least 28° C.

In 2014 an outbreak of a new viral disease infecting tomatoes occurred in Israel and Jordan, which spread quickly worldwide. This new disease was identified a new Tobamovirus, called ToBRFV (also known as TBRFV). ToBRFV infection is associated with necrotic lesions on leaves and tomato plants show mild foliar symptoms at the end of the season but strong brown rugose symptoms on fruits, making the fruit unsuitable for consumption. Tobamovirus is a genus in the virus species Virgaviridae that infects plants, including plants of the Solanaceae family, such as tobacco, potato, tomato, and eggplant and are among the most serious threats to vegetable crops in the world. Tobamoviruses are particularly a problem in tomato crops grown in protected environments and are transmitted over long distances through external seed contamination, and mechanically from plant to plant through common culture practices through workers' hands, clothes, tools, and are capable to preserve infectivity in seeds and contaminated soil.

In the battle against Tobamo virus, resistance was introduced in tomatoes by introgression of resistance genes, which provided complete resistance. However, it is highly likely that over time resistance will be broken, since the virus will adapt and evolve resulting in viral breakthrough. Therefore, a continues battle remains to identify resistance genes or combination of genes needed to provide and maintain resistant crops, especially against the new ToBRFV. Furthermore, recent studies in the field seem to indicate that the ToBRFV is capable to infect and cause disease symptoms in previously resistant tomato plant at high temperatures conditions, i.e. above 28° C., resulting increased hypersensitive response in plants and increase crop damage.

The hypersensitive response (HR) is a defence mechanism in plants that is activated upon recognition of a pathogen or other stressor, such as a Tobamovirus infection. The HR is characterized by rapid programmed cell death in the affected tissues, which limits the spread of the pathogen and prevents further damage to the plant. Tomato plants are known to exhibit an HR when infected with Tobamovirus, a genus of viruses that infects a wide range of plants, including tomato. The HR in tomato plants is triggered by the recognition of viral proteins by plant resistance proteins, which initiates a signalling cascade that leads to the activation of cell death. However, research has shown that the HR in tomato plants can be compromised at high temperatures of 28 degrees Celsius or higher, especially in the case of Tobamovirus infection. This is likely because the heat stress induced at high temperatures can interfere with the signalling pathway that triggers the HR, allowing the virus to spread more easily and cause more damage to the plant. It is believed that the high temperature inhibits the function of the resistance proteins, preventing them from recognizing the viral proteins and initiating the signaling cascade. Additionally, heat stress can also induce the production of heat shock proteins, which can interfere with the activation of the HR.

Overall, the HR is an important defense mechanism in tomato plants against Tobamovirus infection, but its efficacy can be compromised under high temperature conditions. Understanding the molecular mechanisms involved in the HR and its regulation under different environmental conditions can help in the development of strategies to improve plant resistance to viral infections, in particular Tobomovirus infection in tomato plants.

Considering the above, there is a need in the art for a tomato plant that is resistant to ToBRFV while maintaining its agronomical properties at high temperature conditions, i.e. above 28° C. In addition, there is a need in the art for a method for providing tomato plant that is resistant to ToBRFV while maintaining its agronomical properties, including fruit size, fruit yield and foliar health, at these high temperature conditions.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a plant of the S. lycopersicum species that is resistant to Tobamovirus, wherein the plant comprises a TBRFV resistance gene that encodes for a TBRFV resistance protein, wherein the protein has at least 90%, preferably at least 95%, more preferably at least 98%, even more preferably at least 99%, most preferably 100% amino acid sequence identity with SEQ ID No. 2, wherein the plant further comprises at least one temperature sensitivity reducing (TSR) gene that encodes for a TSR protein having at least 95%, more preferably at least 98%, even more preferably at least 99%, most preferably 100% sequence identity with the TSR protein selected from the group consisting of SEQ ID No. 4 (also referred to herein as protein of TSR-1), SEQ ID No. 6 (also referred to herein as protein of TSR-2), and SEQ ID No. 8 (also referred to herein as protein of TSR-3), preferably SEQ ID No. 4.

The present invention relates to a tomato plant that comprises in its genome a combination of a TBRFV gene and a Temperature Sensitivity Reducing (TSR) gene for reducing the hypersensitive response (HR) response of tomato plants at high temperatures >28° C. of at least 24 h, preferably at least 48 h, when contacted with a Tobamovirus, preferably TBRFV. This HR response is associated with unwanted effects such as leaf discoloration, dark spots at tomato fruits, reduced plant viability and eventually yield loss. Surprisingly, it was found that when the ToBRFV resistance gene was combined with the TSR gene a significant improved disease resistance was obtained at high temperatures, in comparison to plants that do not have the TSR gene (but do comprise the ToBRFV gene). It was furthermore shown that the homozygous presence of the TBRFV resistance gene itself has a favourable effect on reducing HR response per se. Experiments show that in case the TBRFV resistance gene in combined with at least of the recited TSR genes, the viral disease resistance is improved, especially in high temperature conditions, i.e, wherein the plant is resistant at a temperature of at least 28° C., preferably at least 30° C., more preferably at least 32° C., most preferably at least 35° C., wherein said temperature is an average temperature that is present for at least 1 subsequent day, preferably for at least 2 subsequent days, more preferably at least 3 subsequent days, even more preferably at least 4 subsequent days, most preferably at least 6 subsequent days. Temperatures can vary within these subsequent days (i.e. higher day time and lower night time temperatures) but, on average would be at least 28° C. with a variation of at most 1 to 7° C. between high and low temperatures providing the average temperature. Furthermore, the temperature cannot drop below 27° C. making up the average temperature.

No disease symptoms of TBRFV infection and/or necrosis could be observed in plants comprised of the combination of the TBRFV and TSR resistance genes. Plants comprising the TBRFV gene, but absent of the TSR gene, seem to be affected by TBRFV infection at such high temperature conditions. The TBRFV and TRG gene seem to synergistically enhance the plants ability to remain fully resistant to the TBRFV infection in high temperature conditions and do not show necrosis, i.e. >28° C. for at least 6 subsequent days.

According to a preferred embodiment, the present invention relates to the plant, wherein the TBRFV resistance gene comprises a coding sequence that has at least 90%, preferably at least 95%, more preferably at least 98%, even more preferably at least 99%, most preferably 100% nucleotide sequence identity with SEQ ID No. 1.

According to yet another preferred embodiment, the present invention relates to the plant, wherein the TBRFV resistance gene is heterozygous or homozygous present in the genome of said plant. From the experimental data it can be concluded that the resistance is dominant and that the TBRFV resistance gene is preferably at least heterozygous present in the genome of the plant to provide resistance against the TBRFV Tobamo virus. A similar picture was seen in the experiments in respect to the one or more TSR gene(s). In case the one or more TSR gene(s) were homozygously present, the resistance against the TBRFV virus of the plant under high temperature conditions was significantly improved.

According to another preferred embodiment, the present invention relates to the plant, wherein the at least one TSR gene is heterozygous or homozygous present in the genome of said plant, preferably homozygous. For example, the plant of present invention comprises the TBRFV gene (homozygous or heterozygous) and/or the TSR-1 gene (homozygous or heterozygous), and/or the TSR-2 gene (homozygous or heterozygous), and/or the TSR-3 gene (homozygous or heterozygous), and/or the TSR-4 locus (homozygous or heterozygous).

According to another preferred embodiment, the present invention relates to the plant, wherein the plant comprises the TBRFV and the TSR gene comprising the coding sequence of SEQ ID No. 2 (TBRFV coding sequence) and SEQ ID No. 4 (TSR-1 coding sequence), respectively, wherein both said genes are homozygous present.

According to yet another preferred embodiment the present invention relates to the plant, wherein the plant comprises the TBRFV gene comprising the coding sequence of SEQ ID No. 2 (TBRFV), and a TSR gene comprising the coding sequence of SEQ ID No. 4 (TSR-1), and a locus (referred to herein as TSR4 locus) comprising another TSR gene (referred to herein as TSR4), wherein the TSR4 gene is linked to the marker sequences of SEQ ID No. 10, SEQ ID No. 11 and SEQ ID No. 12, and preferably wherein all said genes and locus are homozygous present. Results have shown that, in case the plant comprises the genes encoding the coding sequences of SEQ ID No. 2, and SEQ ID No. 4, is further comprising the TSR4 locus, which is linked to the marker sequences of SEQ ID No. 10 (forward marker: TGTTAGGATGCGATATATGTAC), SEQ ID No. 11 (reverse marker, CTGGATAGGAGTGGATTTAC) and SEQ ID No. 12 (probe, GTTTATGTGTTAtGTTGCTATtAATGAGG), a further improved effect on disease resistance to TBRFV and reduces/no necrosis at high temperatures is observed. These improved disease and necrosis phenotypes are also observed in plants comprising the TBRVF gene (i.e. SEQ ID No. 2) and the TSR-4 locus comprising the TSR4 gene (linked to the marker sequences of SEQ ID No. 10, SEQ ID No. 11 and SEQ ID No. 12), and even further improved in combination with SEQ ID No. 4 present in said plant. Preferably said improved plants comprising TBRVF, TSR-1 and TSR-4 genes and locus homozygous in their genome, whereas homozygous plants outperform heterozygous plants in this respect.

According to a preferred embodiment the present invention relates to the plant, wherein the TSR4 locus which is linked (is co-segregating with) to the marker sequences of SEQ ID No. 10, SEQ ID No. 11 and SEQ ID No. 12 and comprises the TSR-4 gene. The TSR4 locus is preferably found as present between position 8,500,000 bp and 10,500,000 bp (Heinz 2.40 reference genome). More preferably the TSR-4 locus is obtainable from the Solanum pimpinellifolium plant PI 79532 (as also described by Andrus C F, Reynard G B, Wade B L (1942) “Relative resistance of tomato varieties, selections, and crosses to defoliation by Alternaria and Stemphylium. US Department of Agriculture Circular 652).

According to a preferred embodiment, the present invention relates to the plant, wherein the plant is resistant to Brown Rugose Fruit Virus (TBRFV), Tobamo virus strains Tm0, Tm1, Tm2, preferably TBRFV.

According to a preferred embodiment, the present invention relates to the plant, wherein the plant is resistant at a temperature of at least 28° C., preferably at least 30° C., more preferably at least 32° C., most preferably at least 35° C., wherein said temperature is an average temperature that is present for at least 1 subsequent day, preferably for at least 2 subsequent days, more preferably at least 3 subsequent days, even more preferably at least 4 subsequent days, most preferably at least 6 subsequent days, and wherein the plant is resistant to TBRFV and does not show Tobamovirus disease and/or necrotic symptoms. As indicated above, the temperature can vary within these subsequent days (i.e. higher day time and lower night time temperatures) but, on average is at least 28° C. with a variation of at most 1 to 7° C. between high and low temperatures providing the average temperature. Furthermore, the temperature cannot drop below 27° C. making up the average temperature within said subsequent days.

According to another preferred embodiment, the present invention relates to the plant, wherein the plant is an agronomical plant that produces agronomical fruits. The plant of present invention maintains one or more agronomical properties selected from the group consisting of fruit size, fruit yield and foliar health, at these high temperature conditions.

According to yet another preferred embodiment, the present invention relates to the plant, wherein said TBRFV resistance gene is as found in the deposit accession number NCIMB 43279. NCIMB 43279, Solanum lycopersicum seeds were deposited on 16 Nov. 2018 at NCIMB Ltd, Ferguson Building, Craibstone Estate Bucksburn, AB21 9YA Aberdeen, United Kingdom.

The present invention, according to a second aspect, relates to plants, plant parts, tissues, cells, and/or seeds derived from a plant as disclosed above.

The present invention, according to a further aspect, relates to a TBRFV resistance gene in combination with at least one temperature sensitivity reducing (TSR) gene in a S. lycopersicum plant for providing resistance to a Tobamovirus in said plant, wherein said TBRFV gene comprises a coding sequence of SEQ ID No. 1 and the TSR gene comprises a coding sequence selected from the group consisting of SEQ ID No. 3 (also referred as TSR-1), SEQ ID No. 5 (also referred to as TSR-2), and SEQ ID No. 7 (also referred to as TSR-3), preferably SEQ ID No. 3. The at least one TSR gene may comprise the TSR 4 locus comprising another TSR gene (referred to herein as TSR4), wherein the TSR4 gene is linked to the marker sequences of SEQ ID No. 10, SEQ ID No. 11 and SEQ ID No. 12. Preferably all (TBRFV and TSR) said genes and locus are homozygous present in the plant of present invention.

According to yet another preferred embodiment, the present invention relates to the TBRFV resistance gene in combination with at least one temperature sensitivity reducing (TSR) gene, wherein the plant is resistant at a temperature of at least 28° C., preferably at least 30° C., more preferably at least 32° C., most preferably at least 35° C., wherein said temperature is an average temperature that is present for at least 1 subsequent day, preferably for at least 2 subsequent days, more preferably at least 3 subsequent days, even more preferably at least 4 subsequent days, most preferably at least 6 subsequent days, and wherein the plant is resistant to TBRFV and does not show Tobamovirus disease and/or necrotic symptoms.

The present invention, according to a further aspect, relates to a method for producing and/or selecting of a plant of the S. lycopersicum species that is resistant to TBRFV, wherein the method comprises the steps of;
a) selecting a first tomato plant, wherein said selection comprises establishing in said first tomato plant the presence of a TBRFV resistance gene that encodes for a TBRFV resistance protein, wherein the protein has at least 90% amino acid sequence identity with SEQ ID No. 2,
b) transferring, for example by crossing, the identified TBRFV resistance gene into a second tomato plant, wherein said second tomato plant comprises at least one TSR gene that encodes for a TSR protein having at least 95% sequence identity with the TSR protein selected from the group consisting of SEQ ID No. 4, SEQ ID No. 6, and SEQ ID No. 8, preferably SEQ ID No. 4.

The present invention, according to a further aspect, relates to a method for selecting a plant of the S. lycopersicum species that is resistant to TBRFV as disclosed herein, wherein the method comprises the selection a S. lycopersicum plant, wherein said selection comprises establishing in said S. lycopersicum plant the presence of a TBRFV resistance gene that encodes for a TBRFV resistance protein, wherein the protein has at least 90% amino acid sequence identity with SEQ ID No. 2, and establishing in said S. lycopersicum plant the presence of a TSR gene that encodes for a TSR protein having at least 95% sequence identity with the TSR protein selected from the group consisting of SEQ ID No. 4, SEQ ID No. 6, and SEQ ID No. 8, preferably SEQ ID No. 4.

The present invention, according to a further aspect, relates to a method for improving the yield and/or reducing the loss on tomato production of a tomato plant in condition of TBRFV infestation, wherein the plant is resistant at a temperature of at least 28° C., preferably at least 30° C., more preferably at least 32° C., most preferably at least 35° C., wherein said temperature is an average temperature that is present for at least 1 subsequent day, preferably for at least 2 subsequent days, more preferably at least 3 subsequent days, even more preferably at least 4 subsequent days, most preferably at least 6 subsequent days, and wherein the plant is resistant to TBRFV and does not show Tobamovirus disease and/or necrotic symptoms, the method comprises the step of providing a plant of the S. lycopersicum species of present invention.

The present invention, according to a further aspect, relates to a method of protecting a field, tunnel, greenhouse or glasshouse of tomato plants from TBRFV infestation, growing a plant of the S. lycopersicum species of present invention.

The present invention, according to a further aspect, relates to the use of at least one temperature sensitivity reducing (TSR) gene in a tomato plant for reducing necrosis at a temperature of at least 28° C., preferably at least 30° C., more preferably at least 32° C., most preferably at least 35° C., wherein said temperature is an average temperature that is present for at least 1 subsequent day, preferably for at least 2 subsequent days, more preferably at least 3 subsequent days, even more preferably at least 4 subsequent days, most preferably at least 6 subsequent days, and wherein the plant is resistant to TBRFV and does not show Tobamovirus disease and/or necrotic symptoms, wherein the at least one TSR gene comprises a coding sequence having at least 95% sequence identity with the sequence selected from the group consisting of SEQ ID No. 3, SEQ ID No. 5, and SEQ ID No. 7, preferably SEQ ID No. 3.

The present invention, according to a further aspect, relates to use of at least one temperature sensitivity reducing (TSR) gene in a tomato plant for reducing the temperature sensitivity of said tomato plant, and/or for improving the vigour of said tomato plant at a temperature of at least 28° C., wherein said temperature is an average temperature that is present for at least 1 subsequent day, preferably for at least 2 subsequent days, and wherein the at least one TSR gene comprises a coding sequence having at least 95% sequence identity with the sequence selected from the group consisting of SEQ ID No. 3, SEQ ID No. 5, and SEQ ID No. 7, preferably SEQ ID No. 3. Further preferably the invention relates to said use of at least one temperature sensitivity reducing (TSR) gene in a tomato plant as indicated above, wherein the tomato plant is resistant to tobamovirus, more preferably TBRFV.

According to a preferred embodiment, the present invention relates to the use wherein the at least one temperature sensitivity reducing (TSR) gene is combined in the tomato plant with a TBRFV gene comprises a coding sequence having at least 90% nucleotide sequence identity with SEQ ID No1.

According to yet another preferred embodiment, the present invention relates to the use wherein the TBRFV resistance gene is heterozygous or homozygous present in the genome of said plant, preferably homozygous, and/or wherein the at least one TSR gene is heterozygous or homozygous present in the genome of said tomato plant, preferably homozygous.

The present invention will be further detailed in the following examples and FIGURES wherein:

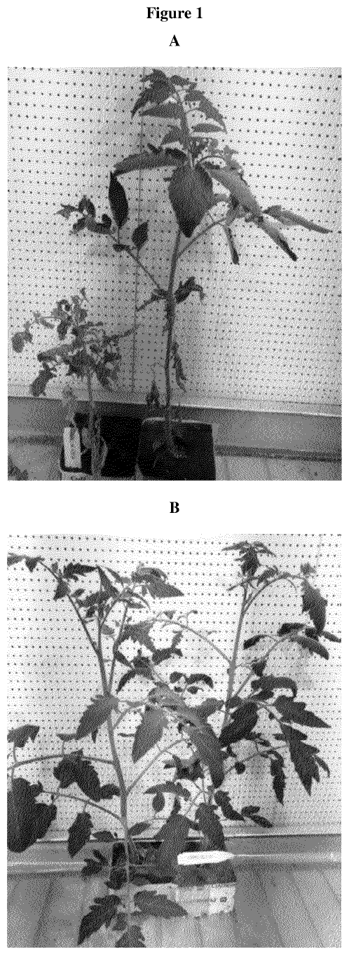

FIG. 1: Tomato plants were treated by heat treatment of at least 6 days at 35° C., followed by a period of 4 to 5 days at 22° C., after which the plants were scored for disease symptoms. Three hours prior to the start of the heat treatment, plants were inoculated with the TBRFV virus. In the FIGS. 1A and 1B, the plants on the left are infected, and on the right are not infected with TBRFV. FIG. 1A shows tomato plants comprised of the TBRFV gene (SEQ ID No. 1, heterozygous) and absent of temperature sensitivity reducing (TSR) gene. FIG. 1B shows tomato plants comprised of both the TBRFV (homozygous) and the TSR gene (SEQ ID No. 3, homozygous). The synergistic effect of the TSR gene in combination with the TBRFV after heat treatment is clearly visible, showing a healthy vigorous plant in case both TBRFV and TSR are present in the plant.

### EXAMPLES

Inoculation of a Tomato Plant with TBRFV

The TBRFV isolate AE050 (Origin Saudi Arabia) was used to perform the disease assays. As plant material, the Line OT9, which is a plant line susceptible for TBRFV was used for virus maintenance. Symptomatic leaves received from the original samples were used for sap-mechanical inoculation on the Line OT9. The virus was maintained on systemically infected tomato plants OT9 by monthly sap-mechanical inoculation on new 3 weeks-old seedlings.

Seeds of tomato plants to be infected were sown in vermiculite, seedlings were transplanted in rockwool blocks and inoculated at 4 weeks after sowing. As starting material symptomatic leaves from infected-OT9 were collected and ground in a mortar and pestle in chilled demi water with carborundum (1 gr/100 mL). The oldest leaf from 3 weeks-old seedlings of each test plant was mechanically inoculated with AE050 by gently rubbing the leaf once with one finger. The plants where phenotyped by visual scoring of the plants and the leaves. The presence of yellowing, mosaic pattern on leaves, leaf deformation (narrowing, mottling) was recorded, see below. Furthermore, qPCR tests on plants were done to determine virus infection.

Determination of TBRFV Infection in Tomato (S. lycopersicum) by qPCR

qPCR was used for determining the viral infection in tomato plants comprising the TBRFV resistance gene and/or a TSR gene (both heterozygous or homozygous). Plants were infected with TBRFV and the susceptible tomato line OT9 has been included as control (OT9). After 3 weeks of inoculation, one leaf punch (6 mm) from the top of the plant of every single plant was collected in a 4 ml Micronic tube which contain a 4 mm metal bullet. 2 ml of extraction buffer (GH+ buffer containing 25 mM TCEP) was added and the tubes were shaken with high speed (1300 rpm) in a seed shaker duo machine to pulverize the plant material. After centrifugation of 20 min at 4,000 g, the RNA extraction was carried out using a Hamilton Microlab Star machine. One-step RT-qPCR (taqman) was conducted using a primer-probe combination for amplifying TBRFV, see Table 1 (SEQ ID No. 11 to 13).

TABLE 1

| qPCR primer name | Sequence |
|---|---|
| TBRFV-Fw | GGTGGTGTCAGTGTCTGTTT |
| (SEQ ID No. 12) | |
| TBRFV-Probe HEX-BHQ1 | AGAGAATGGAGAGAGCGGACGAGG |
| (SEQ ID No. 13) | |
| TBRFV-Rev | GCGTCCTTGGTAGTGATGTT |
| (SEQ ID No. 14) | |

The more viral RNA present in the samples the lower the Ct value in the qPCR, since less PCR cycles are required to amplify the cDNA (of the viral RNA) and pick up a signal. Within this experiment, a control sample (OT9 uninfected) shows a Ct value of about 25 or more cycles. The infected control sample (OT9 infected) in general shows a Ct value between 5 to 15, here a Ct value of 8.9 was observed. Therefore, plants that show a Ct value above 22 cycles are considered resistant, whereas plants that show a Ct value below 15 were considered susceptible, see Table 3.

Tomato plants comprising the TBRFV resistance gene and/or a TSR gene, either homozygous (P) or heterozygous (P) all shown a Ct value above 20 cycles and are considered as resistant. In case any of the genes were homozygous present, an improved resistance was observed, as shown by a higher Ct value. Plants that did not comprise the TBRFV resistance gene and the TSR gene (A) showed a Ct value of 8.9, indicating that the plant was fully susceptible to TBRFV infection.

Evaluate Disease Resistance after Heat Treatment on TBRFV Infected Tomato Plants Comprising the TBRFV Resistance Gene and/or the TSR-1 Gene

Plants having the various genotypes, i.e. comprising one or more of the resistance genes of TBRFV (SEQ ID No. 1) and TSR (SEQ ID No. 3), were treated by heat treatment of at least subsequent 6 days at 35° C. on average (average temperature of day and day and night and temperature does not drop below 27° C.), followed by a period of 3 to 4 days at 22° C., after which the plants were scored for disease symptoms and sampled for RT-qPCR. Three hours prior to the start of the heat treatment, plants were inoculated with the TBRFV virus.

For the heat treatment, plants were placed on benches in a climate cell with Son-T lights. Plants were grown in rockwool blocks and subjected to the heat treatment at an age of 4 weeks (3 leaf stage). Temperatures were set to a specific temperature, but due to the heat produced by the Son-T lights (220V, 400 W), temperatures could deviate about 3 degrees ° C. above or below the set temperature (which would reflect a more natural temperature profile inside a greenhouse). Lights were on for 12 hours per day (from 7:00 to 19:00).

The susceptible tomato line OT9 has been included as control. Plants were categorized as “resistant” when no such symptoms on leaves were observed. Plants displaying any of the symptoms on leaves were categorized as “susceptible”. Leaf samples were collected from asymptomatic plants (i.e resistant) to test for the presence of virus by qPCR (Ct value). The plants where phenotyped by visual scoring of the plants and the leaves. The presence of yellowing, mosaic pattern on leaves, leaf deformation (narrowing, mottling) was recorded. Necrosis was scored on a scale of 1 to 9; 1 showing heavy necrosis and 9 showing no necrosis symptoms, see Table 2 below.

TABLE 2
visual scoring

| Necrosis score | Necrosis symptoms |
|---|---|
| 9 | No symptoms |
| 7 | Necrosis on inoculated leaf |
| 5 | Separate necrotic spots on systemic leaf |
| 3 | Heavy necrosis on systemic leaf |
| 1 | Heavy necrosis on systemic leaf + collapse |

The average of 12 plants is taken to ascertain the necrosis score per plant group tested. Tomato plants comprising the TBRFV gene (SEQ ID No. 1), the TSR gene (SEQ ID No. 3) either homozygously or heterozygously, and combinations thereof, were included in the screening of disease resistance and necrosis after heat treatment on TBRFV infected tomato plants. Furthermore, phenotypic results were confirmed by qPCR (Ct Value; average of 6 plants) to relatively quantify viral loads in the infected plants, see Table 3.

TABLE 3

| | Genotype | | | |
|---|---|---|---|---|
| Plant | TBRFV | TSR-1 | Necrosis score | Ct Value |
| 0 (OT9) | A | A | 1.0 | 8.9 |
| 1 | H | A | 5.0 | 22.2 |
| 2 | H | H | 6.8 | 24.5 |
| 3 | P | A | 5.4 | 23.5 |
| 4 | P | P | 9.0 | 25.4 |

A = absent, H = heterozygous and P = homozygous

From the data it can be concluded that in case the plant comprises the TBRFV gene, at high temperatures (>28° C.) for at least 2-3 days, the plant response is hypersensitive to a TBRFV infection. However, in case the plant further comprises the TSR-1 gene, this hypersensitive response is strongly reduced, or even absent in case the TSR-1 gene is present homozygous in the plant. TSR gene seems to be semi-dominant, since also the presence of heterozygous TSR-1 gene in combination with ToBRFV shows reduced/absent necrosis and improved disease response at high temperatures. Plants comprised of homozygous ToBRFV gene, homozygous TSR-1 gene did not show any disease or necrotic symptoms after 10 days of heat treatment as described above and being inoculated with ToBRFV. Furthermore, plants comprised of ToBRFV gene and the TSR-4 locus comprising the TSR4 gene also showed improved necrosis and improved disease response (data not shown).

1. A tomato plant of the S. lycopersicum species that is resistant to Tobamovirus, wherein the tomato plant comprises a TBRFV resistance gene that encodes for a TBRFV resistance protein, wherein the protein has at least 90% amino acid sequence identity with SEQ ID No. 2, wherein the plant further comprises at least one temperature sensitivity reducing (TSR) gene that encodes for a TSR protein having at least 95% sequence identity with the TSR protein selected from the group consisting of SEQ ID No. 4, SEQ ID No. 6, and SEQ ID No. 8, preferably SEQ ID No. 4.

2. The tomato plant according to claim 1, wherein the TBRFV resistance gene comprises a coding sequence that has at least 90% nucleotide sequence identity with SEQ ID No. 1.

3. The tomato plant according to claim 1, wherein the TBRFV resistance gene is heterozygous or homozygous present in the genome of said plant, preferably homozygous.

4. The tomato plant according to claim 1, wherein the at least one TSR gene is heterozygous or homozygous present in the genome of said plant, preferably homozygous.

5. The tomato plant according to claim 1, wherein the plant comprises the TBRFV and the TSR gene comprising the coding sequence of SEQ ID No. 2 and SEQ ID No. 4, respectively, wherein both said genes are homozygous present.

6. The tomato plant according to claim 1, wherein the plant is resistant to Brown Rugose Fruit Virus (TBRFV), Tobamo virus strains Tm0, Tm1, Tm2, preferably TBRFV.

7. The tomato plant according to claim 1, wherein the plant is resistant at a temperature of at least 28° C., at least 30° C., at least 32° C., or at least 35° C., wherein said temperature is an average temperature that is present for at least 1 subsequent day, for at least 2 subsequent days, at least 3 subsequent days, at least 4 subsequent days, or at least 6 subsequent days, and wherein the tomato plant is resistant to TBRFV and does not show Tobamovirus disease and/or necrotic symptoms.

8. The tomato plant according to claim 1, wherein the plant is an agronomical plant that produces agronomical fruits.

9. The tomato plant according to claim 1, wherein the TBRFV resistance gene is as found in the deposit accession number NCIMB 43279.

10. A plant part, tissue, plant cell, and/or seed derived from the tomato plant according to claim 1.

11. A TBRFV resistance gene in combination with at least one temperature sensitivity reducing (TSR) gene in a S. lycopersicum tomato plant for providing resistance to a Tobamovirus in said tomato plant, wherein said TBRFV gene comprises a coding sequence of SEQ ID No. 1 and the TSR gene comprises a coding sequence selected from the group consisting of SEQ ID No. 3, SEQ ID No. 5, and SEQ ID No. 7, or SEQ ID No. 3.

12. The TBRFV resistance gene in combination with at least one temperature sensitivity reducing (TSR) gene according to claim 11, wherein the tomato plant is resistant at a temperature of at least 28° C., at least 30° C., at least 32° C., or at least 35° C., wherein said temperature is an average temperature that is present for at least 1 subsequent day, for at least 2 subsequent days, at least 3 subsequent days, at least 4 subsequent days, or at least 6 subsequent days, and wherein the tomato plant is resistant to TBRFV and does not show Tobamovirus disease and/or necrotic symptoms.

13. A method for producing a tomato plant of the S. lycopersicum species that is resistant to TBRFV according to claim 1,
wherein the method comprises the steps of:
a) selecting a first tomato plant, wherein said selection comprises identifying in said first tomato plant the presence of a TBRFV resistance gene that encodes for a TBRFV resistance protein, wherein the protein has at least 90% amino acid sequence identity with SEQ ID No. 2; and
b) transferring the identified TBRFV resistance gene into a second tomato plant, wherein said second tomato plant comprises at least one TSR gene that encodes for a TSR protein having at least 95% sequence identity with the TSR protein selected from the group consisting of SEQ ID No. 4, SEQ ID No. 6, and SEQ ID No. 8, preferably SEQ ID No. 4.

14. A method for selecting a tomato plant of the S. lycopersicum species that is resistant to TBRFV according to claim 1, wherein the method comprises the selection of a S. lycopersicum tomato plant, wherein said selection comprises establishing in said S. lycopersicum tomato plant the presence of a TBRFV resistance gene that encodes for a TBRFV resistance protein, wherein the protein has at least 90% amino acid sequence identity with SEQ ID No. 2, and establishing in said S. lycopersicum tomato plant the presence of a TSR gene that encodes for a TSR protein having at least 95% sequence identity with the TSR protein selected from the group consisting of SEQ ID No. 4, SEQ ID No. 6, and SEQ ID No. 8, preferably SEQ ID No. 4.

15. A method for reducing the loss of tomato production of a tomato plant in condition of TBRFV infestation, wherein the tomato plant is resistant at a temperature of at least 28° C., wherein said temperature is an average temperature that is present for at least 1 subsequent day, or for at least 2 subsequent days, the method comprises the step of providing a tomato plant of the S. lycopersicum species according to claim 1.

16. Use of at least one temperature sensitivity reducing (TSR) gene in a tomato plant for reducing necrosis symptoms at a temperature of at least 28° C., wherein said temperature is an average temperature that is present for at least 1 subsequent day, or for at least 2 subsequent days, and wherein the at least one TSR gene comprises a coding sequence having at least 95% sequence identity with the sequence selected from the group consisting of SEQ ID No. 3, SEQ ID No. 5, and SEQ ID No. 7.

17. Use according to claim 16, wherein the at least one temperature sensitivity reducing (TSR) gene is combined in the tomato plant with a TBRFV gene, wherein the TBRFV gene comprises a coding sequence having at least 90% nucleotide sequence identity with SEQ ID No. 1.

18. Use according to claim 16, wherein the tomato plant is heterozygous or homozygous for the TBRFV gene and/or wherein the tomato plant is heterozygous or homozygous for the at least one TSR gene.


SEQUENCE LISTING (14 sequences)

SEQ ID NO: 1 - DNA, 2940 bp, Solanum lycopersicum
  source 1..2940: mol_type = other DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcgcaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaagaaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatggggacattgcaaccatatgaattgtcaaatttgtctcaagaagattgttggttgttgttcatgcaatgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagatagtgagatttggaatttacctcaagatgaaagttctattctgcctgccctgagacttagttaccatcaccttccacttgatttgagacaatgcttttcatattgtgcagtattcccaaaggataccaaaatggaaaaggaaaatctaatctctctctggatggcacatggttttcttttatcaaaaggaaacttggagctagaggatgtaggtaatgaagtatggaatgaattatacttgaggtcttttttccaagagattgaagttcaatatgatcgaacttatttcaagatgcatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgttttccaaaagaaacaagtaaacttggtagtctccgaaatctcttacttgatggttgcgatggattggattctatgccaccaaggataggatctttgacatgccttaagactctaagtttatttgttattggcgagagaaaagattctctacttggtgaattacgaaacctgaatctgtatgggtcaattgaaatcacgcatcttgagagagtgaagaatgatagggatgcaaaagaagccaatttatctgcaaaagaaaatctgcattctttaagcatgagatgggaaggaccacatagatatgaatcagaagaagttgaagtgcttgaatccctcaaaccacactccaatgtgacttgtttaacaatcactggcttcagaggaatccgtctcccagagtggatgaatcactcagttttgaaaaatgttgtctctattgcaattagaggttgtgaaaactgctcatgcttaccaccgtttggtgatctgccttgtctagaaagtctagagttacggagtgggtctgcggaagtggagtatgttgaagattctggattcccaacaagaagaaggtttccatctatgagaaaacttactatagaaaattttgataatctgaaaggattgctgaaagaggcaggagaagagcaattccccgtgcttgaagagttgacaattagatgttgtcctgtgtttgttattccgaccctttcttctgtcaagaaattggtagttcatgggaacaagtcagatgcaatagttttgaggtccatatataatcttagggctcttacttccctcaacattagccataacttcacagctacttcgctcccagaagagatgttcaaaagccttgcaaatctcaaatacttggaaatcgctttcatctccaatctcaaagagctgccaaacagcctggctagtctcaatgctttgaagcatctgtttattaattgttgttttgcactagagagtctccccgaggaagcggtgaaaggtttaacttcactcacacagttatccataacatactgtaagaggctaaaatgtttaccagagggattgcagcaactaacaaatttatcagttaggtattgtccaacactggccaagcgatgtgagaagggaataggacaagactggtacaaaattgctcacattcctcatctgctgattactgattag

SEQ ID NO: 2 - protein, 979 aa, Solanum lycopersicum
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRRKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKKIVESIEEKSLGDMDLAPLQKKLQDLLNGKKYLLVLDDVWNEDQDKWAKLRQVLKAGASGAYVLTTTRLEKVGSIMGTLQPYELSNLSQEDCWLLFMQCAFGHQEEMNLNLVAIGKVIVKKCGGVPLAAKTLGGILRFKREERQWEHVRDSEIWNLPQDESSILPALRLSYHHLPLDLRQCFSYCAVFPKDTKMEKENLISLWMAHGFLLSKGNLELEDVGNEVWNELYLRSFFQEIEVQYDRTYFKMHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCFPKETSKLGSLRNLLLDGCDGLDSMPPRIGSLTCLKTLSLFVIGERKDSLLGELRNLNLYGSIEITHLERVKNDRDAKEANLSAKENLHSLSMRWEGPHRYESEEVEVLESLKPHSNVTCLTITGFRGIRLPEWMNHSVLKNVVSIAIRGCENCSCLPPFGDLPCLESLELRSGSAEVEYVEDSGFPTRRRFPSMRKLTIENFDNLKGLLKEAGEEQFPVLEELTIRCCPVFVIPTLSSVKKLVVHGNKSDAIVLRSIYNLRALTSLNISHNFTATSLPEEMFKSLANLKYLEIAFISNLKELPNSLASLNALKHLFINCCFALESLPEEAVKGLTSLTQLSITYCKRLKCLPEGLQQLTNLSVRYCPTLAKRCEKGIGQDWYKIAHIPHLLITD

SEQ ID NO: 3 - DNA, 2265 bp, Solanum lycopersicum
  source 1..2265: mol_type = other DNA
atggcaactgcacagagtaattctcctcgagttttctgtatcggaacagctgatacaaaattcgacgagcttcgtttcctctccgagcatgtgagatccagtcttaacagcttctccaataaatcctcattcaaggtaggagtgacagttgttgatgtctcaaccagctggaaggagacaaatagttgtgctgattttgattttgtaccgagtaaggatgtgctgtcatgccatacactaggggaagaaactatgggcacgtttgcagatattagaggcctagctattgcaatcatgagcaaagctcttgaaactttcctaagtatagctaatgatgaacagaatcttgctggagtaattggccttgggggtagtggaggaacatctctattgtcatctgccttccgatctcttccaattgggatcccaaaagttataatatctacagttgccagtggtcaaactgaatcttatattggaacatcagacttggtattgtttccttcagttgtagatatttgtgggattaacaatgtcagtaaggttgttctatctaatgcgggtgcagcatttgctggaatggtgatcgggaggcttgaaagttcaaaagagcatagcatcactaatggaaagtttacagttggtgtaactatgtttggggttacgactccttgtgttaatgctgtcaaagaaagattagtgaaagaaggatatgagactttggtgttccatgccacgggtgtcgggggcagggccatggaggatcttgttagaggaggttttatacagggtgtgctggatattacgacaactgaggttgcagattacgtagttggaggagtaatggcatgtgatagttcccgatttgatgcaatattagagaagaaaattcctttggttctgagtgtgggagcactggatatggtgaattttggtcctaaaactaccatacctcctgagtttcaacaaagaaagatccatgaacataatgagcaggtttccctaatgcgtactacagtaggtgaaaataagaaatttgctgcatttatagcagaaaagttgaacaaggcatcatcaagtgtatgtgtttgcttgccagagaaaggcgtgtctgcattggatgcacccgggaaagacttttatgatcctgaggcaactagttgtcttacacgtgaactacagatgcttcttgaaaataatgaacgttgtcaggttaaggtcctcccttaccatatcaatgatgcggagtttgcaaatgctttagttgattcattcttggaaatctctccgaaatctagacacgtagaatgtcagccagctgagtccaaatctatccaagacattcagaatgataatgctgttctagagaaatatccctcatgcaacgggaaaaacttttctcgcctgaatgactttccaaatgcaaaaccagaaactttgcagaaaagaactgtgatactgcagaaattgaaagatcaaataagtaagggcaagcctattattggggctggtgctggtacaggtatttctgctaagtttgaggaagctggtggtgtagatttgattgtcttgtacaactcagggcgctttaggatggcaggaaggggatccttagctggtctactgccctttgctgatgcaaatgccattgtacttgagatggccaacgaagtattgcctgtggttaaggaagtggcagttctggctggagtttgtgctactgatcctttccgcaggatggacaacttcctgaagcagttggaatccgttggattctgtggggtgcaaaactttccaactgttggtctgtttgacggtaacttcagacaaaatttggaagagactggaatgggttatggcttggaggttgagatgattgcagcagctcacaggatgggccttttgacaaccccatatgctttctgcccagatgaagcagttgctatggcagaagctggtgccgacatcatagttgctcatatggggcttacaacatctggttcaattggtgcaaaaacagccgtctcattggaggaaagtgtaacttgcgttcaagctattgcagatgctactcataggatatatcctgatgcaattgtgctctgccatggaggccctatatcttcccctgaagaagcagcatatgtactgaagagaaccacaggagttcatggattttatggcgcttcaagcatggaaagactaccagttgagcaagctataactgcaactgtccagcagtacaagtctatttctatggagtga

SEQ ID NO: 4 - protein, 754 aa, Solanum lycopersicum
MATAQSNSPRVFCIGTADTKFDELRFLSEHVRSSLNSFSNKSSFKVGVTVVDVSTSWKETNSCADFDFVPSKDVLSCHTLGEETMGTFADIRGLAIAIMSKALETFLSIANDEQNLAGVIGLGGSGGTSLLSSAFRSLPIGIPKVIISTVASGQTESYIGTSDLVLFPSVVDICGINNVSKVVLSNAGAAFAGMVIGRLESSKEHSITNGKFTVGVTMFGVTTPCVNAVKERLVKEGYETLVFHATGVGGRAMEDLVRGGFIQGVLDITTTEVADYVVGGVMACDSSRFDAILEKKIPLVLSVGALDMVNFGPKTTIPPEFQQRKIHEHNEQVSLMRTTVGENKKFAAFIAEKLNKASSSVCVCLPEKGVSALDAPGKDFYDPEATSCLTRELQMLLENNERCQVKVLPYHINDAEFANALVDSFLEISPKSRHVECQPAESKSIQDIQNDNAVLEKYPSCNGKNFSRLNDFPNAKPETLQKRTVILQKLKDQISKGKPIIGAGAGTGISAKFEEAGGVDLIVLYNSGRFRMAGRGSLAGLLPFADANAIVLEMANEVLPVVKEVAVLAGVCATDPFRRMDNFLKQLESVGFCGVQNFPTVGLFDGNFRQNLEETGMGYGLEVEMIAAAHRMGLLTTPYAFCPDEAVAMAEAGADIIVAHMGLTTSGSIGAKTAVSLEESVTCVQAIADATHRIYPDAIVLCHGGPISSPEEAAYVLKRTTGVHGFYGASSMERLPVEQAITATVQQYKSISME

SEQ ID NO: 5 - DNA, 2586 bp, Solanum lycopersicum
  source 1..2586: mol_type = other DNA
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcggatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttttcctcctagatgacatttgggatgtggaaatttgggatgatctgaaacttgtccttcctgaatgtgattcaaaagtcggcagtagaattataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgctcaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagccaactttacggtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 6 - protein, 861 aa, Solanum lycopersicum
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVFLLDDIWDVEIWDDLKLVLPECDSKVGSRIIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLANFTVDRYILAVLKTFPKLRKLKMFICKYNEEKMDLSGEANGYSFPQLEVLHIHSPNGLSEVTCTDDVSMPKLKKLLLTGFHCRISLSERLKKLSK

SEQ ID NO: 7 - DNA, 2586 bp, Solanum lycopersicum
  source 1..2586: mol_type = other DNA
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 8 - protein, 861 aa, Solanum lycopersicum
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHCRISLSERLKKLSK

SEQ ID NO: 9 - DNA, 22 bp, synthetic construct
  source 1..22: mol_type = other DNA
tgttaggatgcgatatatgtac

SEQ ID NO: 10 - DNA, 20 bp, synthetic construct
  source 1..20: mol_type = other DNA
ctggataggagtggatttac

SEQ ID NO: 11 - DNA, 29 bp, synthetic construct
  source 1..29: mol_type = other DNA
gtttatgtgttatgttgctattaatgagg

SEQ ID NO: 12 - DNA, 20 bp, synthetic construct
  source 1..20: mol_type = other DNA
ggtggtgtcagtgtctgttt

SEQ ID NO: 13 - DNA, 24 bp, synthetic construct
  source 1..24: mol_type = other DNA
agagaatggagagagcggacgagg

SEQ ID NO: 14 - DNA, 20 bp, synthetic construct
  source 1..20: mol_type = other DNA
gcgtccttggtagtgatgtt